(19) United States
(12) Patent Application Publication
(10) Pub. No.: US 2021/0008273 A1
(43) Pub. Date: Jan. 14, 2021

(54) DRUG SOLUTION ADMINISTRATION DEVICE

(71) Applicant: TERUMO KABUSHIKI KAISHA, Tokyo (JP)

(72) Inventor: Akira KONDO, Kanagawa (JP)

(73) Assignee: TERUMO KABUSHIKI KAISHA, Tokyo (JP)

(21) Appl. No.: 17/036,346

(22) Filed: Sep. 29, 2020

Related U.S. Application Data
(63) Continuation of application No. PCT/JP2019/013311, filed on Mar. 27, 2019.

(30) Foreign Application Priority Data
Mar. 30, 2018 (JP) 2018-067438

(51) Int. Cl. A61M 5/145; A61M 5/158

(52) U.S. Cl. CPC A61M 5/1452; A61M 5/14248; A61M 5/158

(57) ABSTRACT

A drug solution administration device can prevent deformation resulting in bending of a plunger and a drug solution container during delivery of a drug solution. The drug solution administration device includes: a drug solution container having a cylindrical main body filled with drug solution; a housing that holds the drug solution container; a plunger that expels the drug solution in the drug solution container; and a fixing member that fixes the drug solution container to the housing. The housing includes a chassis having a support surface that supports the bottom surface of the main body of the drug solution container, and the fixing member includes a guide part mounted along the main body of the drug solution container from a side opposite the bottom surface of the main body of the drug solution container in a circumferential direction, and an engagement part engageable with the chassis of the housing.

### CROSS-REFERENCES TO RELATED APPLICATIONS

This application is a continuation of International Application No. PCT/JP2019/013311 filed on Mar. 27, 2019, which claims priority to Japanese Patent Application No. 2018-067438 filed on Mar. 30, 2018, the entire content of both of which is incorporated herein by reference.

### FIELD OF THE INVENTION

The present invention generally relates to a drug solution administration device.

### BACKGROUND DISCUSSION

Conventionally, a syringe pump-type drug solution administration device has been known that administers a drug solution filled in a drug solution container into a living body by a pressing action of a plunger. A drug solution administration device of this type includes a cylindrical drug solution container and a plunger mechanism for expelling the drug solution in the drug solution container (see, for example, Japanese Patent Application Publication No. 09-294807). The drug solution container and the plunger mechanism are housed in a housing and supported by the housing.

### SUMMARY

In the abovementioned drug solution administration device, when the plunger advances in the drug solution container to deliver the drug solution, thrust is generated with the forward movement of the plunger, so that an excessive pressing force may be applied to the plunger and the drug solution container. As a result, the plunger and the drug solution container deform so as to bend with respect to the direction of forward movement of the plunger (the longitudinal direction of the plunger and the drug solution container), which may entail a problem that smooth delivery of the drug solution is inhibited.

The drug solution administration device disclosed here is capable of preventing deformation resulting in bending of a plunger and a drug solution container during delivery of a drug solution.

The drug solution administration device includes: a drug solution container that has a main body having a cylindrical shape and filled with a drug solution; a housing that holds the drug solution container; a plunger that expels the drug solution in the drug solution container; and a fixing member that fixes the drug solution container to the housing, wherein the housing includes a support part having a support surface that supports a bottom surface of the main body of the drug solution container, and the fixing member includes a guide part mounted along the main body of the drug solution container from a side opposite to the bottom surface of the main body of the drug solution container in a circumferential direction, and an engagement part engageable with the support part of the housing.

The drug solution administration device can prevent deformation resulting in bending of the plunger and the drug solution container during delivery of the drug solution.

According to another aspect, a drug solution administration device comprises: a housing possessing an interior and a distal end, a drug solution container containing a drug solution, a plunger positioned in the interior of the housing, and a fixing member that fixes the drug solution container in the housing. The housing includes a support that possesses a support surface facing towards the interior of the housing. The support includes a hole formed in the support surface and extending toward an opposite surface of the support. The drug solution container includes a main body possessing a bottom surface that faces and is in contact with the support surface so that the drug solution container is supported on the support surface of the housing. The drug solution container includes a discharge port at a distal end of the drug solution container from which the drug solution is discharged from the drug solution container, and the discharge port projects distally beyond the distal end of the housing so that the distal end of the drug solution container is positioned outside the housing. The plunger is movable inside and relative to the drug solution container to expel the drug solution in the drug solution container through the discharge port. The fixing member includes an overlying part that overlies a portion of the drug solution container and a leg extending away from the overlying part and positioned in the hole in the support.

In accordance with a further aspect, a drug solution administration instrument comprises: a housing possessing an interior and a distal end, a drug solution container containing a drug solution, a plunger positioned in the interior of the housing, and a fixing member that fixes the drug solution container in the housing. The housing includes a support that possesses a support surface facing towards the interior of the housing, and the support includes a hole formed in the support surface and extending toward an opposite surface of the support. The drug solution container includes a main body possessing a bottom surface that faces and is supported by the support surface so that the drug solution container is supported on the support surface of the housing. The drug solution container includes a discharge port at a distal end of the drug solution container from which the drug solution is discharged from the drug solution container. The plunger is movable inside and relative to the drug solution container to expel the drug solution in the drug solution container through the discharge port. The fixing member includes an overlying part that overlies a portion of the drug solution container and a leg extending away from the overlying part and positioned in the hole in the support. The drug solution administration instrument further comprises a puncture needle positioned outside the housing and projecting from a cannula housing and configured to puncture a living body, and a tube positioned outside the housing and communicating the discharge port at the distal end of the drug solution container with the needle so that as plunger is moved to expel the drug solution from the discharge port of the drug solution container the drug solution is supplied to the needle by way of the tube.

### BRIEF DESCRIPTION OF THE DRAWINGS

FIG. 1 is a side view of a drug solution administration system according to an embodiment.

FIG. 2 is a diagram schematically showing an example of use of the drug solution administration system.

FIG. 3 is a schematic perspective view of a drug solution administration device.

FIG. 4 is a schematic perspective view of a chassis included in a housing and components attached to the chassis.

FIG. 5 is a schematic perspective view of a fixing member.

FIG. 6 is a perspective view showing a state before the fixing member is attached to the chassis.

FIG. 7 is a perspective view showing a way of attaching the fixing member to the chassis.

FIG. 8 is an enlarged view showing a way of attaching the fixing member to the chassis.

FIG. 9 is an enlarged view showing a state after the fixing member is attached to the chassis.

FIG. 10 is a perspective view showing holes formed in the chassis.

### DETAILED DESCRIPTION

Set forth below with reference to the accompanying drawings is a detailed description of an embodiment of a drug solution administration system and drug solution administration device representing examples of the inventive drug solution administration system and drug solution administration device disclosed here. The following description is not intended to limit the technical scope and meaning of terms set forth in the claims of the present application. Further, the dimensional ratios in the drawings are exaggerated for convenience of description, and may differ from the actual ratios.

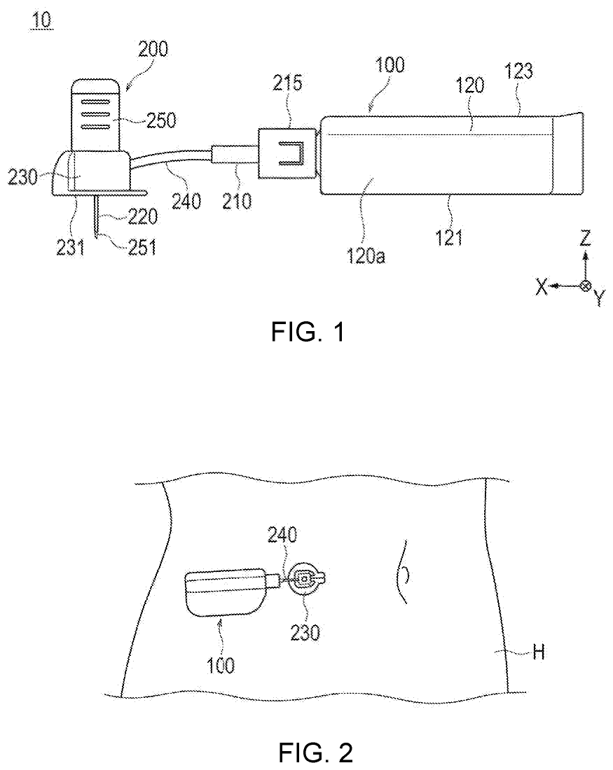

FIGS. 1 to 6 illustrate a drug solution administration system 10, a drug solution administration device 100, and an administration instrument 200 according to the embodiment. An arrow X in each drawing indicates the “longitudinal direction (longitudinal direction of a drug solution container 110)” of the drug solution administration device 100, an arrow Y indicates the “width direction (depth direction)” of the drug solution administration device 100, and an arrow Z indicates the “height direction” of the drug solution administration device 100.

(Drug Solution Administration System)

The drug solution administration system 10 is used to administer a drug solution into a living body. As shown in FIG. 1, the drug solution administration system 10 includes the drug solution administration device 100 and the administration instrument 200.

As shown in FIG. 2, the drug solution administration device 100 and the administration instrument 200 are a patch-type device or instrument that is attached to the body surface (skin) H of a user when used. The body part of the user to which the drug solution administration device 100 and the administration instrument 200 are attached is not particularly limited, and examples of the body part include the abdomen and the thigh.

The drug solution administration system 10 can continuously administer a drug solution filled in the drug solution container 110 included in the drug solution administration device 100 into a living body for relatively a long period of time (for example, several minutes to several hours) by a pressing action of a later-described plunger 130. The drug solution administration system 10 may administer the drug solution at intervals into the living body.

(Drug Solution Administration Device)

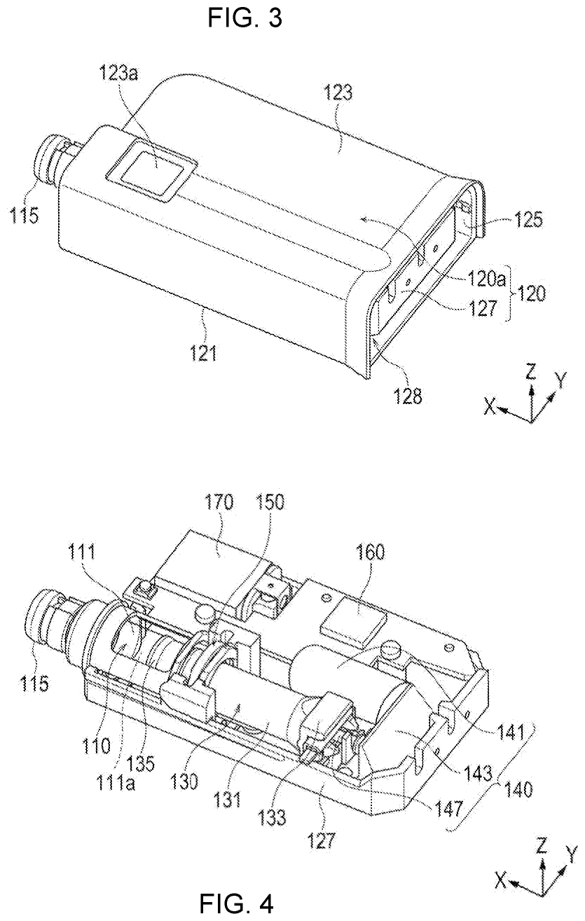

As shown in FIGS. 3 and 4, the drug solution administration device 100 includes the drug solution container 110 having a cylindrical (barrel-shaped) main body 111 filled with a drug solution, a housing 120 for holding the drug solution container 110, a plunger 130 for expelling the drug solution in the drug solution container 110, and a fixing member 150 for fixing the drug solution container 110.

As shown in FIGS. 3 and 4, the housing 120 has a box-shaped housing body 120a having a housing space 128 formed therein, and a chassis 127 (corresponding to a “support” or “support part”) that is housed in the housing space 128 of the housing body 120a and can be fixed to the housing body 120a.

As shown in FIG. 3, a window 123a is formed in an upper surface 123 of the housing body 120a to allow the inside of the housing space 128 to be visible from the outside of the housing 120. The window 123a is formed by providing a transparent or semitransparent portion on a part of the housing body 120a.

A base end opening 125 for inserting the chassis 127 into the housing space 128 of the housing body 120a is formed on the base end side of the housing body 120a in the longitudinal direction. The base end opening 125 of the housing body 120a is closed by a lid member (not shown) with the chassis 127 housed in the housing space 128.

A bottom surface 121 of the housing body 120a is provided with a sheet-shaped adhesive part that can adhere to the body surface H of the user. In an initial state before the drug solution administration device 100 is attached to the user, a releasable protective sheet is attached to an adhesive surface of the adhesive part.

As shown in FIG. 4, the chassis 127 holds the drug solution container 110, the plunger 130, a drive mechanism 140 for driving the forward movement of the plunger 130, a control unit 160, and a power supply unit 170.

A main body 131 of the plunger 130 is inserted into or positioned in an inner cavity 111a of the main body 111 of the drug solution container 110 (see FIG. 4). A gasket 135 is provided at the tip of the main body 131 of the plunger 130. The gasket 135 can be made of, for example, a resin material such as a rubber material or an elastomer. The outer periphery of the gasket 135 is in liquid-tight contact with the inner peripheral surface of the main body 111 of the drug solution container 110, whereby the base end side of the gasket 135 is liquid-tightly sealed.

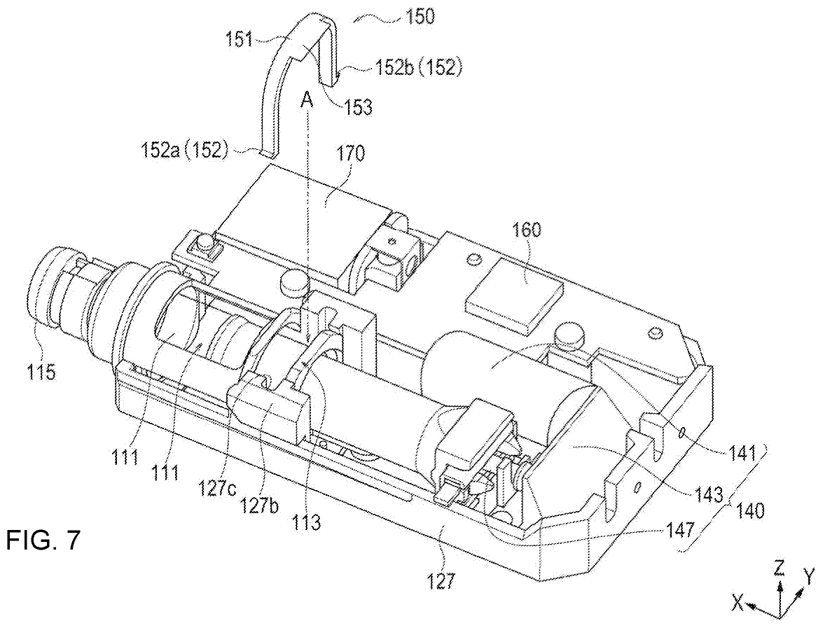

A flange 113 that is wider than the main body 111 of the drug solution container 110 in the radial direction is formed at the base end of the drug solution container 110 (see FIG. 7). The flange is thus a part of the drug solution container 110 that possesses an enlarged outer dimension that is enlarged relative to, or greater than, the outer dimension of the part of the drug solution container 110 immediately adjoining the flange.

The drug solution container 110 is a so-called prefilled type drug solution container. Therefore, the drug solution is filled in advance in the inner cavity 111a of the main body 111 of the drug solution container 110. Examples of the drug solution include protein preparations, narcotic analgesics, and diuretics.

A sealing member (not shown) for preventing the drug solution from leaking is disposed at a tip opening (discharge port) of the drug solution container 110. As shown in FIG. 3, the drug solution container 110 is disposed such that the tip opening projects from the housing body 120a to the outside. In addition, a mounting part 115 that is to be connected to a tube 240 (see FIG. 1) described later is attached to a portion of the drug solution container 110 that protrudes from the housing body 120a.

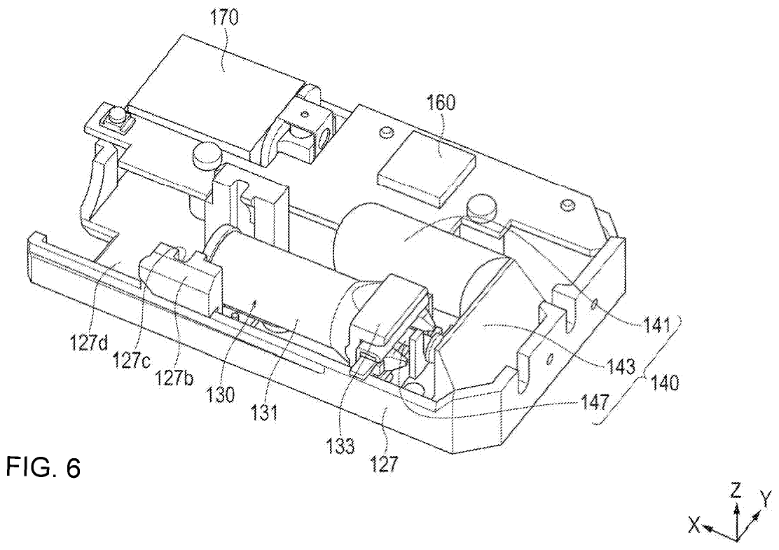

As shown in FIG. 6, the chassis 127 includes a support surface 127d that supports the bottom surface of the main body 111 of the drug solution container 110 (the outer peripheral surface over a certain range located on the chassis 127 side in the circumferential direction). Further, as shown in FIG. 7, the chassis 127 has a flange support part 127b that supports the flange 113 of the drug solution container 110. The flange support part 127b includes a groove 127c into which the flange 113 of the drug solution container 110 can be inserted.

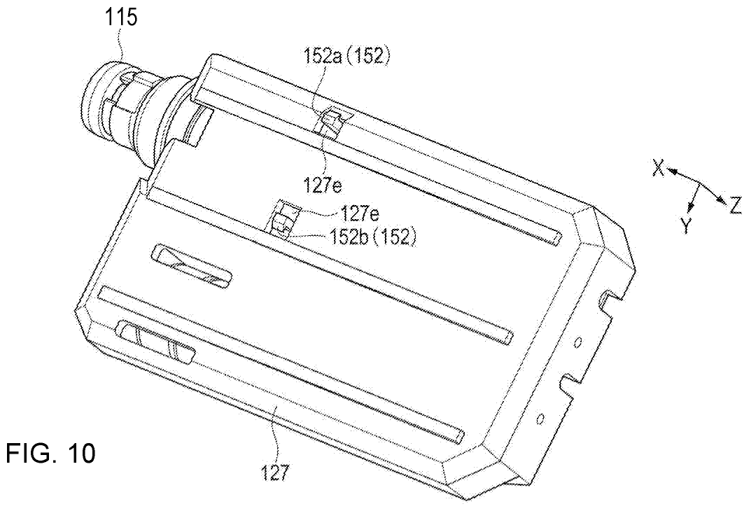

As shown in FIG. 10, the back surface of the chassis 127 (the surface reverse to the surface having the support surface 127d on which the drug solution container 110 and the like are placed) has holes 127e into which tabs (transversely projecting tabs) 152a and 152b of the fixing member 150 described later can be fitted.

The control unit 160 controls the operation of the drug solution administration device 100 for delivering the drug solution. The control unit 160 can be achieved by, for example, a known microcomputer (electronic circuit element) mounted with a CPU, a RAM, a ROM and the like.

The power supply unit 170 can be composed of, for example, a known battery.

As shown in FIG. 4, the drive mechanism 140 includes a motor 141 that receives a drive current from the power supply unit 170 to generate a rotational drive force, a reduction mechanism 143 that includes a gear that transmits the rotational drive force of the motor 141, and the like, and a feed screw 147 connected to the reduction mechanism 143.

The feed screw 147 is connected to a base end connection part 133 disposed near the base end of the plunger 130. The feed screw 147 converts the rotary motion transmitted from the reduction mechanism 143 into a linear motion, and advances the plunger 130 in the longitudinal direction (X direction). The plunger 130 expels the drug solution from the inner cavity 111a of the main body 111 of the drug solution container 110 to the tube 240 (see FIG. 1) due to the movement of the plunger toward the tip of the drug solution container 110.

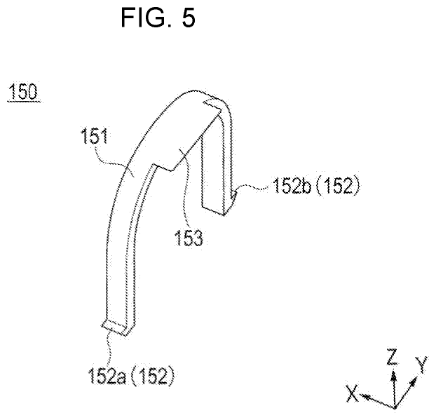

Next, the fixing member or fixing bracket 150 will be described with reference to FIG. 5.

As shown in FIG. 5, the fixing member 150 includes a guide part 151 and an engagement part 152. The guide part 151 is mounted along the main body 111 of the drug solution container 110 from the side reverse or opposite to the bottom surface of the main body 111 of the drug solution container 110 in the circumferential direction (from an upper surface side reverse to the surface of the drug solution container 110 facing the chassis 127). The engagement part 152 is engageable with the chassis 127.

The guide part 151 of the fixing member 150 has a curved shape so as to be attached along the outer peripheral surface of the main body 111 of the drug solution container 110. Further, the guide part 151 has a protrusion 153 protruding in a predetermined direction.

The engagement part 152 of the fixing member 150 has a first tab 152a and a second tab 152b. As shown in FIG. 10, the first tab 152a and the second tab 152b are configured to be engageable with the holes 127e formed in the chassis 127.

As shown in FIG. 9, while the flange 113 of the drug solution container 110 is inserted or positioned in the groove 127c of the flange support part 127b of the chassis 127, the guide part 151 of the fixing member 150 is mounted on the tip side of the flange 113 so as to hold the flange 113 with the flange support part 127b (with the surrounding inner wall of the groove 127c).

Next, a procedure for mounting the fixing member 150 will be described.

First, as shown in FIG. 6, the components other than the drug solution container 110 are attached to the chassis 127.

Next, as shown in FIG. 7, the drug solution container 110 is attached to the chassis 127. At this time, the bottom surface of the main body 111 of the drug solution container 110 is placed on the support surface 127d of the chassis 127. Then, as shown in FIG. 7, the fixing member 150 is mounted to the chassis 127. As indicated by an arrow A in FIG. 7, the fixing member 150 is mounted to the chassis 127 from the side opposite to the side where the bottom surface of the drug solution container 110 is placed.

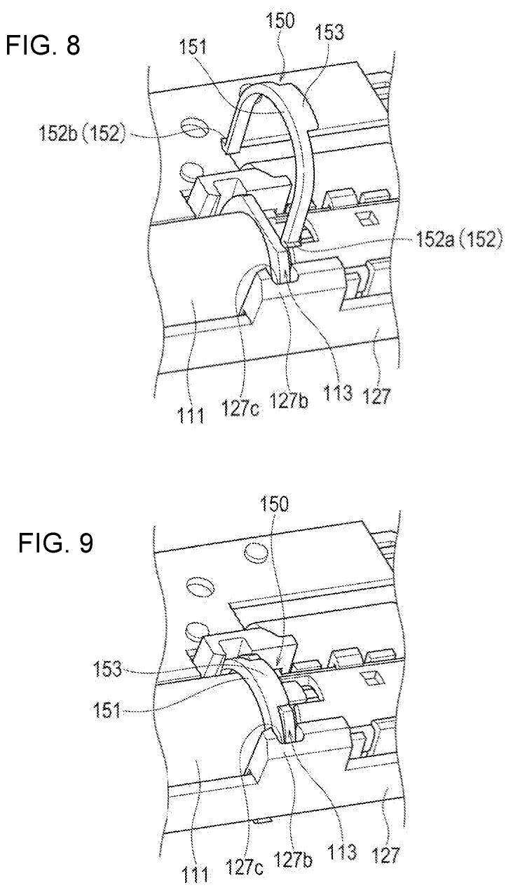

When the fixing member 150 is mounted, the guide part 151 of the fixing member 150 is mounted along the outer surface of the main body 111 of the drug solution container 110 as shown in FIGS. 8 and 9. Further, the guide part 151 is mounted on the tip side of the flange 113. The protrusion 153 of the fixing member 150 is mounted or positioned so as to cover or overlie the flange 113 of the drug solution container 110 from the upper surface side of the flange 113.

As shown in FIG. 10, the fixing member 150 is fixed to the chassis 127 by engagement between the tabs 152a and 152b and the holes 127e formed in the chassis 127. The drug solution container 110 is fixed to the chassis 127 by means of the fixing member 150. That is, as shown in FIGS. 7 and 8, the fixing member includes a curved upper portion that overlies the flange 113 as well as the drug solution container 110, and two spaced apart legs that project away from the curved upper portion towards the support surface 127d.

While the drug solution administration device 100 is delivering the drug solution, the bottom surface of the main body 111 of the drug solution container 110 is supported by the chassis 127, and the upper surface of the main body 111 of the drug solution container 110 is fixed or held by the fixing member 150. Therefore, even when the plunger 130 and the drug solution container 110 are excessively pressed because of thrust generated due to the forward movement of the plunger 130 within the drug solution container 110, the drug solution administration device 100 can press the plunger 130 and the drug solution container 110 in a direction intersecting the longitudinal direction of the drug solution container 110 (direction orthogonal to the direction of the forward movement of the plunger 130). Therefore, the drug solution administration device 100 can adequately prevent the drug solution container 110 and the plunger 130 from bending in an inverted V shape.

(Administration Instrument)

As shown in FIGS. 1 and 2, the administration instrument 200 is connectable to the drug solution administration device 100.

The administration instrument 200 includes a connector 210, a needle tube (needle) 220 that is inserted into a living body, a puncture part (cannula housing) 230, a tube 240, and a puncture assistance tool 250 that assists the puncture of the living body with the needle tube 220.

The connector 210 is connectable to the drug solution administration device 100 via a mounting part 215 fixed to the connector 210. The mounting part 215 can be connected to the drug solution administration device 100 by being externally fitted to the mounting part 115 (see FIG. 7) which is provided near the tip of the drug solution container 110 and protrudes to the outside of the housing 120.

The mounting part 215 has inside a connecting needle (not shown) capable of piercing or configured to puncture/pierce a sealing member (not shown) provided at the tip of the drug solution container 110. The tube 240 communicates with the inner cavity 111a of the main body 111 of the drug solution container 110 via the connecting needle.

A flow path that connects or fluidly communicates the tube 240 and the inner cavity of the needle tube 220 is formed inside the puncture part 230. The drug solution delivered to the puncture part 230 via the tube 240 is administered into the living body through the flow path formed inside the puncture part 230 and the needle tube 220.

When the drug solution is delivered to the user, the puncture assistance tool 250 is attached to the puncture part 230. The puncture assistance tool 250 holds an introducer needle (inner needle) 251. The introducer needle 251 projects from the tip of the needle tube 220 when the puncture assistance tool 250 is attached to the puncture part 230. The user can insert the needle tube 220 into the living body, while preventing the needle tube 220 from having any troubles such as breakage, by piercing or puncturing the living body with the needle tube 220 with the introducer needle 251 inserted into and projecting from the needle tube 220.

The puncture assistance tool 250 is removed from the puncture part 230 after the needle tube 220 is inserted into the living body. The introducer needle 251 is withdrawn from the inner cavity or lumen of the needle tube 220 when the puncture assistance tool 250 is removed from the puncture part 230.

After the needle tube 220 is inserted into the living body, the puncture assistance tool 250 is removed, and the puncture part 230 is left on the body surface H of the user with the needle tube 220 left in the living body. In this state, the plunger 130 of the drug solution administration device 100 advances in the drug solution container 110, so that the drug solution filled in the drug solution container 110 is delivered to the inner cavity or lumen of the needle tube 220 through the tube 240 and the flow path of the puncture part 230.

The introducer needle 251 can be, for example, a metal needle. Further, the needle tube 220 can be composed of, for example, a tubular member (cannula) made of resin.

Similar to the drug solution administration device 100, the administration instrument 200 is of a patch type that is attached to the body surface H of the user when used. The contact surface (bottom surface) 231 of the puncture part 230 of the administration instrument 200 is provided with a sheet-shaped adhesive part (not shown) that can adhere to the body surface. In an initial state before the administration instrument 200 is attached to the user, a releasable protective sheet is attached to an adhesive surface of the adhesive part.

As described above, the drug solution administration device 100 according to the present embodiment includes the drug solution container 110 having the cylindrical main body 111 filled with a drug solution, the housing 120 for holding the drug solution container 110, the plunger 130 for expelling the drug solution in the drug solution container 110, and the fixing member 150 for fixing the drug solution container 110 to the housing 120. The housing 120 includes the chassis 127 having the support surface 127d that supports the bottom surface of the main body 111 of the drug solution container 110. The fixing member 150 includes the guide part 151 that is mounted along the main body 111 of the drug solution container 110 from the side reverse to the bottom surface of the main body 111 of the drug solution container 110 in the circumferential direction, and the engagement part 152 that is engageable with the chassis 127 of the housing 120.

The drug solution administration device 100 configured as described above delivers the drug solution, while the bottom surface of the main body 111 of the drug solution container 110 is supported by the chassis 127, and the upper surface of the main body 111 of the drug solution container 110 is fixed by the fixing member 150. Therefore, it is possible to prevent deformation resulting in bending of the plunger 130 and the drug solution container 110 during delivery of the drug solution.

Further, in the drug solution administration device 100, the guide part 151 of the fixing member 150 has a curved shape so as to be attached along the outer peripheral surface of the main body 111 of the drug solution container 110. Therefore, the fixing member 150 can rigidly fix the outer peripheral surface of the main body 111 of the drug solution container 110 by the guide part 151.

Further, in the drug solution administration device 100, the chassis 127 included in the housing 120 has the holes 127e, and the engagement part 152 of the fixing member 150 has the tabs 152a and 152b that can be fitted into and engage the holes 127e. Therefore, the fixing member 150 can be easily and reliably fixed to the chassis 127 by means of the tabs 152a and 152b.

Further, in the drug solution administration device 100, the flange 113 that is wider than the main body 111 of the drug solution container 110 in the radial direction is formed at the base end of the drug solution container 110. The chassis 127 included in the housing 120 has the flange support part 127b that supports the flange 113 of the drug solution container 110. Further, the guide part 151 of the fixing member 150 is mounted on the tip side of the flange 113 so as to hold the flange 113 of the drug solution container 110 with the flange support part 127b. Due to the flange 113 of the drug solution container 110 being held between the fixing member 150 and the flange support part 127b as described above, the fixing member 150 can more reliably fix the drug solution container 110 to the chassis 127.

While the drug solution administration device has been described above by way of the embodiment, the present invention is not limited to each of the described configurations, and can be appropriately modified on the basis of the description of the claims.

For example, the specific shape, structure, and the like of the fixing member are not particularly limited, as long as the fixing member has a guide part and an engagement part, and is configured to fix the main body of the drug solution container to the housing. Further, for example, the engagement part can be configured to be engageable with the housing by a structure other than the structure using the tabs described in the embodiment. In addition, the housing may not include the chassis, and the housing body may be provided with a support part that supports the drug solution container. In this case, the fixing member can be configured to fix the drug solution container to the housing body.

Further, the material, shape, size, or arrangement of the members constituting the drug solution administration device, a structure of connecting/joining the members, and the like are not particularly limited as long as the effects of the present invention are exhibited, and can be optionally changed and replaced. Further, it is possible to appropriately add arbitrary components and the like not particularly described in the specification to the drug solution administration device.

The detailed description above describes an embodiment of a drug solution administration device and drug solution administration system representing an example of the inventive drug solution administration system and drug solution administration device disclosed here. The invention is not limited, however, to the precise embodiment and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A drug solution administration device comprising:
a drug solution container comprising a main body possessing having a cylindrical shape and filled with a drug solution, the main body of the drug solution container possessing a bottom surface;
a housing holding the drug solution container;
a plunger that expels the drug solution in the drug solution container;
a fixing member that fixes the drug solution container to the housing;
the housing including a support part having a support surface that supports a bottom surface of the main body of the drug solution container; and
the fixing member including a guide part mounted along the main body of the drug solution container from a side opposite to the bottom surface of the main body of the drug solution container in a circumferential direction, the fixing member also including an engagement part engageable with the support part of the housing.

2. The drug solution administration device according to claim 1, wherein the guide part has a curved shape so as to be mounted along an outer peripheral surface of the main body of the drug solution container.

3. The drug solution administration device according to claim 1,
wherein the housing has a hole formed in the support part, and
the engagement part of the fixing member has a tab engageable with the hole.

4. The drug solution administration device according to claim 1,
wherein the drug solution container includes a base end, the base end of the drug solution container including a flange that is wider than the main body of the drug solution container in a radial direction,
the housing including a flange support part that supports the flange of the drug solution container, and
the guide part of the fixing member being disposed on a tip side of the flange to hold the flange with the flange support part.

5. The drug solution administration device according to claim 1, wherein the guide part has an upper portion that overlies the drug solution container and two legs extending away from the upper portion towards the support surface, each of the legs possessing a free end, the engagement part being located at the free end of one of the legs.

6. The drug solution administration device according to claim 1, wherein the guide part possesses a curved inner peripheral surface facing a curved outer periphery of the drug solution container.

7. The drug solution administration device according to claim 1, wherein the guide part possesses an inner peripheral surface having a shape that surrounds a part of a curved outer periphery of the drug solution container.

8. The drug solution administration device according to claim 1, wherein the drug solution container includes an enlarged portion possessing an outer dimension larger than an outer dimension of a part of main body of the drug solution container adjoining the enlarged portion, the housing including an upstanding support part provided with a groove, the enlarged portion of the drug solution container being positioned in the groove of the upstanding support part.

9. The drug solution administration device according to claim 8, wherein a part of the fixing member is also positioned in the groove of the upstanding support part.

10. A drug solution administration device comprising:
a housing possessing an interior and a distal end, the housing including a support that possesses a support surface facing towards the interior of the housing, the support including a hole formed in the support surface and extending toward an opposite surface of the support;
a drug solution container containing a drug solution, the drug solution container including a main body possessing a bottom surface that faces and is in contact with the support surface so that the drug solution container is supported on the support surface of the housing, the drug solution container including a discharge port at a distal end of the drug solution container from which the drug solution is discharged from the drug solution container, the discharge port projecting distally beyond the distal end of the housing so that the distal end of the drug solution container is positioned outside the housing;
a plunger positioned in the interior of the housing and movable inside and relative to the drug solution container to expel the drug solution in the drug solution container through the discharge port; and
a fixing member that fixes the drug solution container in the housing, the fixing member including an overlying part that overlies a portion of the drug solution container and a leg extending away from the overlying part and positioned in the hole in the support.

11. The drug solution administration device according to claim 10, wherein the hole in the support is a first hole, the support further comprising a second hole formed in the support surface and extending toward the opposite surface of the support, the leg being a first leg, the fixing member further comprising a second leg extending away from the overlying part and positioned in the second hole in the support.

12. The drug solution administration device according to claim 11, wherein an end of each of the first and second legs remote from the overlying part includes a transversely projecting tab, the transversely projecting tab at the end of the first leg being positioned in the first hole, the transversely projecting tab at the end of the second leg being positioned in the second hole

13. The drug solution administration device according to claim 10, wherein the overlying part possesses a curved inner peripheral surface facing a curved outer surface of the drug solution container.

14. The drug solution administration device according to claim 10, wherein the drug solution container includes an enlarged portion possessing an outer dimension larger than an outer dimension of a part of main body of the drug solution container adjoining the enlarged portion, the housing including an upstanding support part provided with a groove, the enlarged portion of the drug solution container being positioned in the groove of the upstanding support part.

15. The drug solution administration device according to claim 14, wherein a part of the fixing member is also positioned in the groove of the upstanding support part.

16. The drug solution administration device according to claim 10, wherein the hole in the support is a through hole.

17. A drug solution administration instrument comprising:
a housing possessing an interior and a distal end, the housing including a support that possesses a support surface facing towards the interior of the housing, the support including a hole formed in the support surface and extending toward an opposite surface of the support;
a drug solution container containing a drug solution, the drug solution container including a main body possessing a bottom surface that faces and is supported by the support surface so that the drug solution container is supported on the support surface of the housing, the drug solution container including a discharge port at a distal end of the drug solution container from which the drug solution is discharged from the drug solution container;
a plunger positioned in the interior of the housing and movable inside and relative to the drug solution container to expel the drug solution in the drug solution container through the discharge port;
a fixing member that fixes the drug solution container in the housing, the fixing member including an overlying part that overlies a portion of the drug solution container and a leg extending away from the overlying part and positioned in the hole in the support;
a puncture needle positioned outside the housing and projecting from a cannula housing and configured to puncture a living body; and
a tube positioned outside the housing and communicating the discharge port at the distal end of the drug solution container with the needle so that as plunger is moved to expel the drug solution from the discharge port of the drug solution container the drug solution is supplied to the needle by way of the tube.

18. The drug solution administration instrument according to claim 17, further comprising a mounting part mounted on the discharge port, the tube being connected to the mounting part by way of a connector.

19. The drug solution administration instrument according to claim 17, further comprising an introducer needle projecting from a puncture assistance tool, the introducer needle being positioned inside the puncture needle and projecting distally beyond a distal tip of the puncture needle.

20. The drug solution administration instrument according to claim 17, further comprising a motor positioned in the housing and operatively connected to the plunger to move the plunger and expel the drug solution from the discharge port of the drug solution container.
